(19) United States
(12) Patent Application Publication
(10) Pub. No.: US 2025/0360029 A1
(43) Pub. Date: Nov. 27, 2025

(54) MEDICAL DEVICES INCLUDING RELEASE LINERS AND RELEASE LINER TAPE AND/OR HOLDING INDICIA, AND METHODS OF CLOSING A WOUND OR INCISION USING THE MEDICAL DEVICES

(71) Applicant: Clozex Medical, Inc., Franklin, MA (US)

(72) Inventors: Barrett Johnston, Medway, MA (US); Robert Kevin Moore, Boynton Beach, FL (US)

(73) Assignee: Clozex Medical, Inc., Franklin, MA (US)

(21) Appl. No.: 19/209,290

(22) Filed: May 15, 2025

Related U.S. Application Data
(60) Provisional application No. 63/651,012, filed on May 23, 2024.

(51) Int. Cl. A61F 13/02; A61F 13/00

(52) U.S. Cl. CPC A61F 13/0243; A61F 13/00059; A61F 13/0259

(57) ABSTRACT

Medical devices including release liners and release liner tape and/or holding indicia, and methods of closing a wound or incision using the medical devices.

### BACKGROUND

A medical device, for closing wounds or incisions, may include first and second adhesive-backed anchoring members. To close the wound or incision, the anchoring members are adhered to the patient's skin proximate to opposing edges of the wound or incision, and the anchoring members are thereafter drawn toward each other.

The medical device may be packaged with a release liner protecting adhesive-backed surfaces of the anchoring members. Once at least one anchoring member has been peeled away from the release liner, the anchoring members can be freely manipulated by the user to widen, narrow, or angle one anchoring member in relation to the other.

An experienced user may completely remove the release liner and manipulate the anchoring members in a manner that prevents their adhesive-backed surfaces from inadvertently sticking to each other or the patient's skin prior to proper application of the anchoring members to the patient's skin. Alternatively, an experienced user may remove only a first anchoring member from the release liner, thereby exposing only the adhesive-backed surface of that anchoring member, allowing the user to place the first anchoring member at a desired position with respect to a first edge of the wound or incision. Subsequently, the user may remove the remainder of the release liner from the second anchoring member and place it proximate at a desired position with respect to an opposing, second edge of the wound or incision. Thereafter, the user may draw the two anchoring members toward each other to close the wound or incision.

A new (e.g., inexperienced or first-time) user, however, may render the medical device unusable by improperly handling the medical device during or after the release liner is removed from one or both of the anchoring members. For example, the new user may inadvertently permit the exposed adhesive-backed surfaces of the anchoring members to stick to each other or, while adhering one anchoring member to the patient's skin, may inadvertently allow the opposing anchoring member to stick to the patient's skin at an undesired position with respect to the wound or incision.

### SUMMARY

A first aspect of the present disclosure relates to a medical device for closing a wound or incision, comprising: first and second components that are movable with respect to each other for closing a wound or incision; a first release liner removably adhered to a first adhesive-backed surface of the first component; a second release liner removably adhered to a second adhesive-backed surface of the first component and/or a first adhesive-backed surface of the second component; and means located on the first release liner and the second component for maintaining a relative position of the first and second components while and after the second release liner is removed from the medical device.

In at least some embodiments of the first aspect, the means does not extend to any edge of the second component.

In at least some embodiments of the first aspect, the means is not located on the second release liner.

In at least some embodiments of the first aspect, the means is a release liner tape.

In at least some embodiments of the first aspect, the release liner tape extends beyond at least one edge of the second component.

In at least some embodiments of the first aspect, a portion of the release liner tape, extending beyond the at least one edge of the second component, does not have any adhesive thereon.

In at least some embodiments of the first aspect, a portion of the release liner tape, extending beyond the at least one edge of the second component, has a deadened adhesive thereon.

In at least some embodiments of the first aspect, the release liner tape extends beyond an edge of the second component oriented orthogonal to a wound edge of the second component.

In at least some embodiments of the first aspect, the release liner tape extends beyond an edge of the second component oriented parallel to a wound edge of the second component.

In at least some embodiments of the first aspect, the release liner tape decrease in width as it moves toward an edge of the medical device.

In at least some embodiments of the first aspect, the release liner tape is configured to remove the first release liner from the medical device when the release liner tape is removed from the medical device.

In at least some embodiments of the first aspect, a bottom surface of portion of the release liner tape, covering a portion of a pulling element of the first component, either lacks adhesive thereon or has a dead zone of adhesive thereon.

In at least some embodiments of the first aspect, a portion of the release liner tape is covered by a tab of the second release liner when the tab is oriented towards and planar with a pulling element of the first component; and a bottom surface, of the portion of the release liner tape covered by the tab, either lacks adhesive thereon or has a dead zone of adhesive thereon.

In at least some embodiments of the first aspect, the means is located on the first release liner and a pulling element of the second component, the pulling element being configured to receive a pulling force to close the wound or incision.

In at least some embodiments of the first aspect, the means is indicia printed on the first release liner and the second component.

In at least some embodiments of the first aspect, the first adhesive-backed surface of the first component is on an adhesive-backed anchoring member of the first component, and the adhesive-backed anchoring member is configured to be adhered to skin proximate the wound or incision.

In at least some embodiments of the first aspect, the medical device further comprises a third release liner removably adhered to a second adhesive-backed surface of the second component, wherein the means is located on the first release liner, the second component, and the third release liner.

In at least some embodiments of the first aspect, the second adhesive-backed surface of the second component is on an elongated connector for adhering the first component to the second component and maintaining the wound or incision in a closed state.

In at least some embodiments of the first aspect, the second release liner is removably adhered to the second adhesive-backed surface of the first component and the first adhesive-backed surface of the second component.

In at least some embodiments of the first aspect, the second adhesive-backed surface of the first component is on an adhesive-backed anchoring member of the first component; the first adhesive-backed surface of the second component is on an adhesive-backed anchoring member of the second component; and the adhesive-backed anchoring members are configured to be adhered to skin proximate the wound or incision.

A second aspect of the present disclosure relates to a method for closing a wound or incision, comprising providing a medical device of the first aspect; applying the medical device to skin of a patient proximate the wound or incision; and after applying the medical device, closing the wound or incision using the medical device.

### BRIEF DESCRIPTION OF DRAWINGS

For a more complete understanding of the present disclosure, reference is now made to the following description taken in conjunction with the accompanying drawings.

FIG. 1 is a top view of an interlaced medical device showing slit- or slice-type discontinuities in adhesive-backed anchoring members.

FIG. 2 is a bottom projection view of an interlaced medical device including release liners.

FIG. 3 is a perspective view of an interlaced medical device showing a first adhesive-backed anchoring member protected by a first release liner and a second release liner. A second adhesive-backed anchoring member is shown with a mirror image release liner configuration including a first release liner and a second release liner.

FIG. 4a is a perspective view of an example wherein a common release liner is shared between two components of a medical device.

FIG. 4b is a perspective view of an example wherein a crease is provided in a common release liner shared between two components of a medical device.

FIG. 4c is a perspective view of an example wherein a frangible line is provided in a common release liner shared between two components of a medical device.

FIG. 5 is a bottom planar view of a medical device including release liners and a release liner tape. Crosshatching used for visualization purposes.

FIG. 6 is a bottom planar view of a medical device including release liners and a release liner tape. Crosshatching used for visualization purposes.

FIG. 7 is a bottom planar view of a medical device including release liners and a release liner tape. Crosshatching used for visualization purposes.

FIG. 8 is a bottom planar view of a medical device including release liners and a release liner tape. Crosshatching used for visualization purposes.

FIG. 9 is a bottom planar view of a medical device including release liners and a release liner tape. Crosshatching used for visualization purposes.

FIG. 10 is a bottom planar view of a medical device including release liners and a release liner tape. Crosshatching used for visualization purposes.

FIG. 11 is a bottom planar view of a medical device including release liners and a release liner tape. Crosshatching used for visualization purposes.

FIG. 12 is a bottom planar view of a medical device including release liners and a release liner tape. Crosshatching used for visualization purposes.

FIG. 13 is a bottom planar view of a medical device including release liners and a release liner tape. Crosshatching used for visualization purposes.

FIG. 14 is a bottom planar view of a medical device including indicia for holding a release liner(s) while removing another release liner. Crosshatching used for visualization purposes.

FIG. 15 is a bottom planar view of a medical device including indicia for holding a release liner(s) while removing another release liner. Crosshatching used for visualization purposes.

FIG. 16 is a process flow diagram illustrating a method for closing a wound or incision.

### DETAILED DESCRIPTION

The present disclosure provides, among other things, medical devices having at least two release liners and at least one release liner tape attached to the release liners in a manner that maintains a relative positioning of first and second components of the medical device to promote proper removal of at least one release liner from the medical device. First and second components of the medical device may each have a release liner thereon, and the release liner tape may be removably coupled to the two release liners and the pulling element in a manner that maintains a relative position of the first component with respect to the second component. The release liner tape may also be removably coupled to the release liners in a manner that enables a third release liner of the medical device to be removed without changing the relative position of the first component with respect to the second component in an undesired way.

The present disclosure also provides, among other things, medical devices having indicia for holding the medical device in a manner that maintains a relative positioning of the medical device's first and second components to promote proper removal of at least one release liner from the medical device. The indicia may be located on aspects of the first and second components to facilitate a user to simultaneously grasp aspects of the first and second components of the medical device and thereby maintain a relative position of the first component with respect to the second component.

A medical device and release liner tape of the present disclosure may have some or all of the features described here.

### Medical Devices

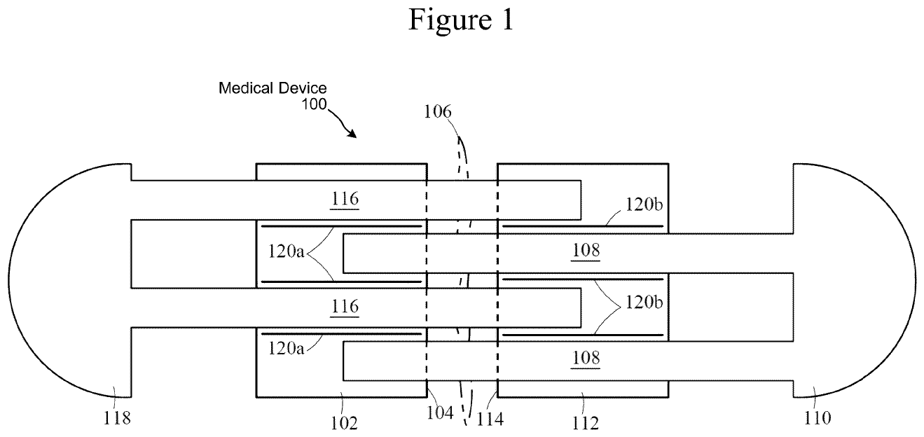

A medical device of the present disclosure may include at least two opposing adhesive-backed anchoring members that are movable with respect to each other and designed for placement with respect to opposing edges of a wound or incision (i.e., designed for adhesion to patient skin proximate to opposing edges of a wound or incision) such that drawing the adhesive-backed anchoring members toward each other closes the wound or incision. An example of such a medical device 100 is illustrated in FIG. 1.

The medical device 100 includes a first component including a first adhesive-backed anchoring member 102 having a “wound” or “incision” edge 104 for placement at a desired location with respect to an edge of a wound or incision 106. The first component also includes one or more first elongated connectors 108 extending from the first adhesive-backed anchoring member 102 and a first pulling element 110 joined to the one or more first elongated connectors 108 and adapted for translation of the first adhesive-backed anchoring member 102 toward the wound or incision 106.

The medical device 100 also includes a second component including a second adhesive-backed anchoring member 112 having a “wound” or “incision” edge 114 for placement at a desired location with respect to another edge of the wound or incision 106. The second component also includes one or more second elongated connectors 116 extending from the second adhesive-backed anchoring member 112 and a second pulling element 118 joined to the one or more second elongated connectors 116 and adapted for translation of the second adhesive-backed anchoring member 112 toward the wound or incision 106.

The medical device 100 includes means for attaching the one or more first elongated connectors 108 to the second adhesive-backed anchoring member 112 thereby forming an attached portion and a bridging portion for each attached first elongated connector 108. The medical device 100 also includes means for attaching the one or more second elongated connectors 116 to the first adhesive-backed anchoring member 102 thereby forming an attached portion and a bridging portion for each attached second elongated connector 116.

### Interlaced and Keyhole Embodiments

Embodiments of the medical device 100 can be interlaced or non-interlaced. Interlaced embodiments are those where both the first and second components comprise two or more elongated connectors, where the first and second elongated connectors of the first component are attached to a single pulling element and the first and second elongated connectors of the second component are attached to another single pulling element. Further, an elongated connector from one of the two components passes through the void formed between the two elongated connectors of the other component. When assembled in this manner, the first and second components are linked and cannot be separated without cutting or breaking at least one of the two components. A simple analogy to such an interlaced device is a pair of interlocking rings.

One of skill in the art will recognize that more complex interlaced embodiments can be produced where, referring to a first component, interlacing extends to two or more adjacent voids formed by three or more adjacent elongated connectors. Examples of such embodiments are provided in the disclosures of U.S. Pat. Nos. 6,329,564; 6,831,205; 6,822,133; 7,511,185; 8,636,763; 7,414,168; 7,332,641; 7,354,446; 8,636,763; 7,838,718; 7,414,168; and 7,563,941, the disclosures of which are incorporated herein by reference in their entireties.

Interlaced embodiments can be produced in a variety of ways. Consider, for example, the simplest embodiment to which the analogy to interlocking rings has been made. To produce such an interlaced structure, the first and second components can be die cut as monolithic elements. One of the two components can then be cut, interlaced with the uncut component, and the cut can then be repaired, for example, with adhesive or some other appropriate means. As an alternative method for producing the simplest interlaced embodiment, one component can be die cut monolithically, and the second component can be die cut with an element, or a portion of an element missing. For example, the second component could be die cut with one of the two required elongated connectors being absent. The missing second elongated connector could be provided as a separate die cut element, complete with adhesive. Following interlacing of the first component with the partial second component, the interlaced structure could be completed by attaching the separately supplied missing elongated connector.

A non-interlaced, alternative design may be referred to as a “key-hole” design. In this design, for example, the two components of the medical device 100 are separately produced. In a preferred method, they are produced through a die cut process as monolithic components. Each of the two monolithic components comprise an adhesive-backed anchoring member, elongated connector(s), and a pulling element. The elongated connector(s) of the first component are centrally located in the assembled and applied device. The elongated connectors of the second component are spaced apart thereby creating a void, or “key-hole” through which the pulling element and elongated connector(s) of the first element are inserted. Following insertion and rotation of the first and second components into a common plane, the two components are mated in a “key-hole” arrangement and the medical device is in condition for use.

In some embodiments, the adhesive-backed anchoring members 102/112, elongated connectors 108/116, and pulling elements 110/118 are produced from a substantially inelastic material. In some embodiments, the adhesive-backed anchoring members 102/112, elongated connectors 108/116, and pulling elements 110/118 are produced from a substantially inelastic polymeric material (e.g., polyurethane, polyester, polyethylene, or a combination thereof). Alternatively, they may be produced from an elastic material which is reinforced with an inelastic structural component thereby rendering the medical device substantially inelastic. For example, such inelastic materials may include monofilament polymeric line or mesh. Such reinforced polymers are referred to herein as polymeric composites. A reinforcing, inelastic structural material is referred to in the art as “scrim.” Scrim may be a woven textile or polymer, a non-woven polymer, or any other structural material that acts to stabilize the substrate. In some embodiments, the scrim reinforced substrate may have a high degree of permeability (e.g., 1,000 to 8,000 liters/sec/m²).

Additionally, non-reinforced polymers exhibiting a degree of elasticity (e.g., polyurethane or polyester) may be used in the production of the adhesive-backed anchoring members 102/112 for embodiments in which the adhesive-backed anchoring members 102/112 and elongated connectors 108/116 are produced separately, and subsequently attached to one another (i.e., non-monolithic embodiments). An example polymer for the production of the adhesive-backed anchoring members 102/112 is polyurethane having a thickness of 3-12 mils. Such a polymer is breathable and exhibits a degree of flexibility. If a non-reinforced elastic polymer is used to produce an adhesive-backed anchoring member 102/112, it may be preferable to reinforce the top surface of the wound edge 104/114 with an inelastic element so that the wound edge 104/114 remains substantially straight across the incision site during the closure process. Such an element is referred to herein as a “wound edge bar.” One skilled in the art will recognize that a wide range of inelastic polymers, or even metals, can be utilized in the production of a wound edge bar for the purpose of providing rigidity to the wound edge. Vapor permeable polymeric materials that satisfy the other requirements for use in the manufacturing of the medical device 100 may offer improved comfort. Transparent stock may be preferred so that the healing process and the entire wound site can be monitored easily. Therefore, at least the first and second adhesive-backed anchoring members 102/112 are produced from transparent stock in some embodiments. As an alternative design choice, colored or opaque stock may be used in the production of at least the first and second adhesive-backed anchoring members 102/112 when circumstances (e.g., cost considerations) dictate or permit.

### Sheet Stock

In some embodiments, the adhesive-backed anchoring members 102/112, elongated connectors 108/116, and pulling elements 110/118 are produced from sheets or rolls of polymeric material or polymeric composite material (e.g., polyurethane or polyester). The sheet or roll stock is sometimes referred to as “film” as the thickness in some embodiments ranges from about 0.5 mil to about 5 mil and may vary depending upon application. Die cutting these elements from polymeric sheet stock to provide two monolithic components (i.e., having no seams or joints) which, when assembled/packaged comprise the medical device 100, is a particularly cost-effective approach to manufacturing. Die cutting can be combined with other assembly steps, for example, in connection with the production of interlaced embodiments as discussed elsewhere herein. Laser and ultrasonic trimming devices are also examples of equipment that can be used to cut the components of the medical device 100. The sheet stock may be perforated to allow for the exchange of air with the skin beneath the medical device 100.

### Adhesives

The adhesives selected for use in connection with the medical device 100 may meet a number of requirements. First, adhesive which is to contact the patient's skin may be selected to minimize the potential for adverse reaction by the skin. That is, the adhesive selected may be hypoallergenic. Additionally, all adhesives, whether or not they are intended to contact the skin, should provide a secure hold for a period of time sufficient for the healing process to progress to the point where removal of the medical device 100 is appropriate. An adhesive hold period of about 7-10 days is generally suitable.

Adhesive is a preferred means of attaching one or more elongated connectors 108/116 to an adhesive-backed anchoring member 102/112. In one embodiment, adhesive is applied to at least a portion of the bottom surface of the elongated connectors 108/116 for attaching the elongated connectors 108/116 of one of the two components to the applied adhesive-backed anchoring member 102/112 of the other component. Alternatively or additionally, adhesive may be applied to a portion of the top surfaces of the first and second adhesive-backed anchoring members 102/112. Release liners may be used to protect applied adhesives prior to application of the medical device 100.

Example adhesives include, but are not limited to, tackified acrylic adhesive, pressure-sensitive tackified acrylic adhesive, non-tackified acrylic adhesive, acrylate adhesive, pressure-sensitive acrylate adhesive, natural rubber adhesive, synthetic rubber adhesive, silicone adhesive (repositionable or not), heat-activated adhesive, pressure-sensitive silicone adhesive, transparent adhesive, rubber/acrylic adhesive, or a combination thereof.

### Elongated Connectors

The elongated connectors 108/116 have two parts or portions, an attached portion and a bridging portion. The attached portion of the elongated connectors 108/116, as the name indicates, is that portion which is attached to the adhesive-backed anchoring member 102/112 of the opposing component following application of the medical device 100 to the patient's skin. The bridging portion is the portion of the elongated connector 108/116 which spans over the wound or incision. In some embodiments, the bottom surface of the bridging portion contains less adhesive than the attached portion. In some embodiments, the entire bridging portion is free of adhesive or, alternately, has adhesive but this is blocked with another film (kill layer) to render the adhesive in the bridging portion nonfunctional.

The dimension of the elongated connectors 108/116 may be strap-like in that their width is substantially greater than their thickness. Since the point of attachment between the first and second adhesive-backed anchoring members 102/112 is between the underside/backside of attached portions of elongated connectors 108/116 with the top surface of the adhesive-backed anchoring members 102/112, maximizing the area of contact will result in a more secure closure of the medical device 100 because the area of adhesive contact is maximized. Thus, from the standpoint of security of closure, wider attached portions are preferred. However, as the width of all the elongated connectors 108/116 is increased, the distance between elongated connectors 108/116 necessarily is decreased.

As was stated in U.S. Pat. No. 6,329,564, the disclosure of which is incorporated herein by reference: There is no absolute minimum which can be stated with respect to spacing between elongated connectors 108 or 116. Preferred ranges may be stated as a percentage of device length (i.e., the dimension of the medical device generally parallel the wound or incision). For example, a spacing of between about 5% to about 10% of the medical device length is an example of an appropriate range.

This spacing provides substantial adhesive contact between attached portions of elongated connectors 108/116 with the adhesive-backed anchoring members 102/112. When the wound or incision is closed, the bridging portions of the one or more first elongated connectors 108 and the bridging portions of the one or more second elongated connectors 116 may be aligned with one another over the closed wound or incision. In some embodiments, the average width of the bridging portions is less than the average width of the attached portions of the elongated connectors 108/116. Average width is determined by measuring from the outer perimeters of the bridging portions and the outer perimeters of the attached portions.

This difference in width in the bridging portion relative to the attached portion affords advantages. Consider, for example, a device designed for maximum security. In such a device, the elongated connectors would be placed as close as possible, while still providing for a minimum acceptable degree of adjustment range. If the bridging area were narrowed in such a device, the net effect would be an increase in exposed area over the wound or incision (which is desirable for application of medicines, removal of exudates, etc.), as well as an increase in the range of adjustment (narrowing the width of the elongated connectors in the bridging portion effectively increases the distance between adjacent bridging portions).

Considering the device discussed in the preceding paragraph, holding the width of bridging portion constant, while increasing the width of the attached portions provides for greater security as the area of adhesive contact is effectively increased. It will be recognized by one skilled in the art that hybrid configurations (i.e., devices having narrowed bridging portions and widened attached portions relative to uniform width devices) represent embodiments of the present disclosure.

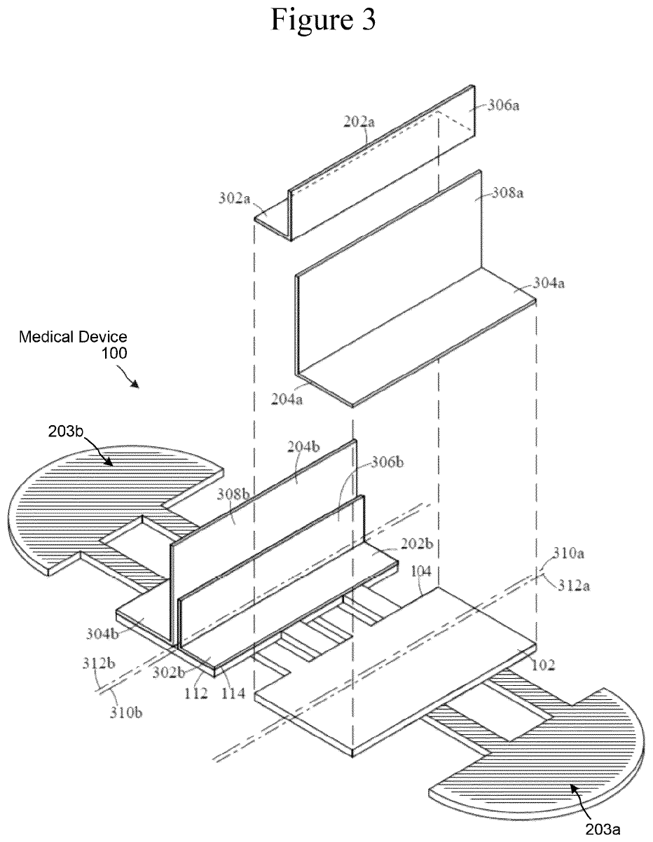

The elongated connectors 108/116 may be viewed as strap-like in their dimensions. In some embodiments, a portion of the elongated connectors 108/116 is cut away to increase the unobstructed surface area over the wound or incision. This tends to facilitate drainage of exudates and application of medication. This cut-out may be best produced during the die cut process. U.S. Pat. No. 6,329,564, the disclosure of which is incorporated herein by reference, depicts cut-outs, for example, in FIG. 3. The shape of the cut-out is not critical. What is important is that the structural integrity of the elongated connectors 108/116 is not compromised by the introduction of the cut-outs.

### Pulling Elements

Embodiments of the present disclosure include pulling elements 110/118 which are attached to elongated connectors 108/116, or to extensions of elongated connectors 108/116. Extensions of elongated connectors 108/116 could themselves be considered pulling elements in embodiments in which only one elongated connector is associated with a component. By definition, the attached portion of an elongated connector 108/116 attaches to the adhesive-backed anchoring member 102/112 of another component of the medical device 100. Extensions of an elongated connector 108/116 may extend the length of the elongated connector 108/116 for case of application, and may be generally removed following the application process. Perforations or scoring may be provided to facilitate their removal. For embodiments in which the number of elongated connectors 108/116 associated with an adhesive-backed anchoring member 102/112 is greater than one, a pulling element 110/118 may be useful for joining the elongated connectors 108/116 or extensions thereof to enable a user to easily apply a pulling force to more than one elongated connector 108/116.

Removal of the pulling elements 110/118 minimizes the footprint of the applied medical device 100. This decrease in the overall size of the medical device 100 reduces the chance that a portion of the medical device 100 may be caught, for example, on clothing or a pillow. Such an occurrence could tend to pull the applied medical device 100 away from the skin thereby causing the wound or incision to open. Minimizing the overall footprint of the applied medical device 100 also tends to provide for a more comfortable fit.

### Eversion Edges

In some embodiments, the wound edges 104/114, of the first and second adhesive-backed anchoring members 102/112, are adapted to evert (or raise) skin edges to promote wound/incision healing. It is known in the art that everting, raising, or mounding of the skin edges at the wound or incision site prevents wound inversion. One way in which this can be accomplished is to provide a bend at the wound edge 104/114. The bend may be angled or arcuate. The adhesive on the lower portion of the adhesive-backed anchoring members 102/112 is also applied to the wound edge portion. When attached to the skin this eversion edge tends to lift the edges of the skin at the point of closure contact, thereby promoting wound or incision healing.

### Coding

To minimize confusion for new users of the medical device 100, the pulling elements 110/118 and adhesive-backed anchoring members 102/112 may be coded to enable user distinction. Thus, for example, the coding may comprise an observable geometric distinction between the shape of the pulling elements 110/118 and the shape of the adhesive-backed anchoring members 102/112. In some embodiments, such coding may comprise printed indicia to enable user distinction between the components. Colors may also be used to provide this distinguishing function.

### Lateral Translation Element

In instances of the present disclosure, both the first and second adhesive-backed anchoring members 102/112 comprise one or more first elongated connectors attached to a single first pulling element. In other instances, one of the first or the second adhesive-backed anchoring members, but not both, comprise one or more lateral translation elements. In these embodiments, one of the first or second adhesive-backed anchoring components comprises one or more lateral translation elements, and the other adhesive-backed anchoring component comprises a plurality of lateral translation elements, each comprising one or more elongated connectors attached to a single pulling element.

Each lateral translation element is selected from the group consisting of: a) a single elongated connector and a single pulling element; and b) a plurality of elongated connectors and a single pulling element.

### Alternative Stock

The embodiments of the medical device 100 discussed above comprise first and second components which are monolithic in nature. That is, the first component (which includes the anchoring member 102 and one or more elongated connectors 108) is produced from a single sheet of stock material without joints or seams. The same statement applies to the second component. In an alternative embodiment, the first and second components are not monolithic in nature. This alternative embodiment is based on the recognition that the desired physical properties of the anchoring members 102/112 and the elongated connectors 108/116 are not, in every instance, identical. For example, a degree of elasticity is a desirable feature in an anchoring member 102/112 when applied, for example, to an area such as a joint. An anchoring member 102/112 produced from a film having a degree of elasticity is less likely to release prematurely than an anchoring member 102/112 produced from a substantially inelastic material when applied to such an area. Elasticity may be a property to be avoided when producing elongated connectors 108/116. Stretching of elongated connectors 108/116 may allow premature opening of a wound or incision.

In embodiments in which the first and second components are not monolithic, the anchoring members 102/112 may be produced from stock having a degree of elasticity. The elongated connectors 108/116 may be produced separately from stock which is substantially inelastic. The one or more first elongated connectors 108 are then attached (e.g., with adhesive) to the first anchoring member 102 to produce a first component. A second component is similarly constructed. As discussed elsewhere, a wound edge bar may be attached to reinforce the wound edge, particularly in embodiments wherein the sheet stock employed has a degree of elasticity.

It is not a requirement that elongated connectors and anchoring members of non-monolithic components be produced from different stock material. It may be desirable, for example, to create an overlap in a portion of the elongated connectors (e.g., the bridging portion) in order to provide for additional strength. Thus, double thickness in the bridging area may be provided by producing a monolithic anchoring member including a portion of a connecting member. A separately produced elongated connector is then attached, in an overlapping manner, to the monolithic anchoring member. This creates a first component which is double-thick in the bridging portion for additional strength and further eliminates stretching.

### Reinforcing Elements

It may be desirable to reinforce the wound edge portion of the anchoring member 102/112 with another layer of less flexible stock. This “wound edge bar” would provide better translation of the force applied by the elongated connectors 108/116 uniformly along the entire wound edge 104/114. Similarly, it may be desirable to reinforce the optional pulling element 110/118, or a portion thereof, with another layer of less flexible stock. This “pull bar” would be useful in applying uniform tension from the pulling element 110/118 to all elongated connectors 108/116, as the medical device 100 is positioned for closure. This feature would become more important in embodiments of the medical device 100 intended to close long wounds or incisions where there might be up to four or more elongated connectors 108/116 to be pulled and secured to each adhesive-backed anchoring member 102/112.

### Elastic Tension Indicators

The medical device 100 may optionally include an elastic tension indicator element. The purpose of the tension indicator element is to provide a visual indication that a desired tension has been reached while applying the medical device 100. For example, materials are known in the art which change color when a predetermined tension is applied. Similarly, other graphic representations may be used for this purpose. For example, a rectangular graphic representation may be applied to an elastic tension indicator element. As this tension indicator is stretched, the graphic representation of the rectangle stretches. This element may be designed such that the desired tension is indicated when the original rectangular representation is stretched to the point where it closely approximates a geometric square.

It may be desirable that this elastic tension indicator element be removable with the pulling elements 110/118 following application of the medical device 100. At a minimum, the elastic tension indicator element should be positioned in the medical device 100 such that when the medical device 100 is applied, it is not possible for the elastic element to continue to stretch and release the desired tension previously established.

### Transdermal Drug Delivery

The medical device 100 can be optionally adapted for transdermal drug delivery. As is known in the art, a drug is deliverable transdermally through the skin. For such an application, a drug-containing patch is secured to at least one of the adhesive-backed anchoring members 102/112 in such a way that the drug can be delivered through the skin. Given the fact that there will be no adhesive contact between the skin and the adhesive-backed anchoring member 102/112 in the area of the drug delivery patch, it may be necessary to increase the size of the anchoring member 102/112 to secure the medical device 100 in such a transdermal drug delivery embodiment. Transdermal drug delivery is well known in the art and a review of the background is not necessary to enable one of skill in the art to make and use the present disclosure.

### Embedded Infection Indicators

Embedded infection indicators can be incorporated to provide for a wound closure device that can, for example, change color as an indication of the presence of unwanted bacteria. One technology utilizes the release of a fluorescent dye from nanocapsules, the release being triggered by toxins secreted by the unwanted bacteria.

### Discontinuities in Adhesive-Backed Anchoring Members

The adhesive-backed anchoring members 102/112 may be characterized by the presence of one or more discontinuities in the polymeric film allowing for the release of sweat from beneath the adhesive-backed anchoring members 102/112. The use of an inherently breathable polymeric film (i.e., a polymeric film, that without any mechanical manipulation, allows for the exchange of air with the skin beneath the adhesive-backed anchoring members 102/112) for the production of the adhesive-backed anchoring members 102/112 may not allow for the release of sweat.

Certain mechanical manipulation of an inherently breathable polymeric film that does not allow for the release of sweat will permit sweat to pass from the skin beneath the medical device 100. The same can be said for mechanical manipulation of a polymeric film that is not inherently breathable.

The introduction of discontinuities that do allow for the release of sweat from beneath the adhesive-backed anchoring members 102/112 addresses the problem of adhesion loss. There is variability in the size, number, and distribution of active sweat glands in humans. For example, according to one expert estimate, the palm of the hand has about 370 sweat glands per cm². By comparison, the back of the hand has about 200; the forehead has about 175; the breast, abdomen, and forearm have about 155; and the leg and back have about 60-80 (all expressed in sweat glands per cm²). Given this sweat gland distribution pattern, one skilled in the art will recognize that discontinuities should be introduced broadly, across the area of the adhesive-backed anchoring members 102/112, to be most effective in addressing the problem of adhesion loss.

Discontinuities can be introduced into the polymeric material used to produce the adhesive-backed anchoring members 102/112 in a variety of ways. It is not a requirement that all discontinuities introduced into a particular adhesive-backed anchoring member be homogenous or uniform. Die cutting technology is a method for the introduction of discontinuities. Die cutting is a process involving the use of a die to shear a web or webs of low-strength materials such as polymeric sheet materials. For example, a needle or pin die could be used to introduce hundreds, or even thousands of small, round discontinuities into the first and second adhesive-backed anchoring members 102/112 through a perforation process. Perforation, as used herein, refers to a process wherein a discrete piercing element, such as a pin in an array of pins assembled on a die, penetrates a material leaving no excess material on either side (e.g., entry or exit side). Whether penetration by a particular piercing element leaves excess material on one side or another (e.g., deformation of material on the exit side) depends not solely on the piercing element, but also the material being pierced (in this case a polymeric sheet material). For example, a larger gauge piercing element, the size of a small nail, for example, may create exit deformation in some polymeric sheet stocks that would not be useful for the introduction of discontinuities consistent with the present disclosure. The shape of a piercing element need not be round. There are no geometric restrictions on the shape of a piercing element.

For the introduction of larger discontinuities, like those produced by piercing a polymeric material with a small nail, a punch process may be more appropriate. A punch, as used herein, is contrasted from a perforation by the requirement that material is removed in a punch process. For example, a punch die of a particular diameter would punch, or remove, a chad from the polymeric material. Such loose chads are removed in a variety of ways in a die cutting process including, for example, inclusion of an adhesive web to capture cut chads, or a vacuum process. Punches will tend to be visible to a user inspecting the applied medical device 100 whereas slits, slices, and perforations are practically invisible to the unaided eye.

In addition to perforation or punching, as described above, cutting (e.g., die cutting) can be used to introduce one or more slits (or slices) 120 into the adhesive-backed anchoring members 102/112. Again, in view of the sweat gland distribution discussed above, a plurality of slits 120 may be preferred.

One of skill in the art will recognize that a certain amount of routine experimentation may be required to optimize discontinuity size, shape, and distribution. Certainly, larger gauge perforations, punches, or slits will allow for the transfer of sweat from the skin beneath the medical device 100. Certain smaller gauge perforations, produced for example using a pin die as discussed above, may allow for vapor transfer but not sweat transfer. It is a matter of routine experimentation to determine discontinuity parameters that will allow for sweat transfer. Optimal discontinuity design for one specific polymer sheet backed with one specific adhesive, may not work well using a different polymer sheet and/or different adhesive. A particularly soft or gummy adhesive, for example, may function in a self-healing role by flowing in to fill perforations when such perforations are introduced with a particularly small diameter piercing element.

It will also be recognized by one of skill in the art that punch-type discontinuities will tend to remove skin contact surface area (and adhesive) from an adhesive-backed anchoring member 102/112. For this reason, larger punch-type discontinuities (e.g., paper punch size discontinuities, or larger) may not be favored, at least for applications requiring high adhesion characteristics.

For a variety of reasons, slits or slices introduced into the adhesive-backed anchoring members may be preferred. For one, like perforations, slits or slices do not remove material from the adhesive-backed anchoring members 102/112 and, therefore, the adhesive-backed anchoring members 102/112 retain their full surface area and adhesive content following the introduction of the slits or slices. Slits or slices can be straight or curvilinear and the slits or slices can be relatively long (e.g. running the length or width of an adhesive-backed anchoring member 102/112) or generally short in length. Furthermore, under flexion, a slit or slice will tend to open up. This tendency serves at least two purposes that represent advantages in the context of the medical device 100. First, the “opening up” of a slit or slice under flexion enables relative unimpeded transfer of sweat from the surface of the skin beneath the adhesive-backed anchoring member 102/112 to the external environment. Second, the “opening up” of the slit or slice tends to allow the adhesive-backed surfaces adjacent to the slit or slice to remain in good adherence with the skin. The presence of the slit or slice tends to reduce peel or shear forces that tend to result in poor adherence characteristics. When introduced, in embodiments of slices that are relatively long the effect can be the creation of a plurality of adhering subdomains in the adhesive-backed anchoring member 102/112.

In some instances, the slits 120 are introduced into each adhesive-backed anchoring member 102/112 in a direction generally perpendicular to the wound edge 104/114 of the adhesive-backed anchoring member 102/112. The slits 120 may be positioned so that they will fall between elongated connectors 108/116. Slits oriented in this way tend to allow a particular adhesive-backed anchoring member 102/112 to be viewed as a unit having a number of adhering subdomains, with the adhering subdomains being divided by the introduced slits. The adhered medical device 100 opens in an accordion-like manner under flexion. The adhering subdomains can actually separate from one another over time thereby creating independent subdomains.

Mechanical manipulation of the type described above can provide for “elastic-like” properties in non-elastic materials. Although the mechanical manipulation does not change the non-clastic characteristic of the particular material, the introduction of voids or discontinuities can allow for movement in dimensions where no movement would be possible absent the voids or discontinuities.

While the length of the slits 120 can be variable, as can the number of slits between elongated connectors 108/116, it may be beneficial that one end of the slits 120 closely approach the wound edge 104/114. It may also be beneficial that the other end of the slits 120 closely approach the edge generally opposite wound edge 104/114. The reason for the stated preference as to the length of the slits 120 is that the combined shear strength of two individual segments of polymeric material exceeds that of an otherwise identical single polymeric segment construct having equal area as compared to the two individual segments combined. The effective separation of adhesive-backed anchoring member 102/112 into individual segments (also referred to as adhering subdomains), as shown in FIG. 1 will provide for enhanced shear strength as compared to the adhesive-backed anchoring member 102/112, lacking the slits 120.

It was stated above that it may be beneficial that one end of the slits 120 closely approach the wound edge 104/114. The integrity of the wound edge 104/114 should be maintained during the application process. Therefore, prior to application, the slits 120 should not extend all the way to the wound edge 104/114, thereby severing the wound edge 104/114. In another instance of the present disclosure, the slits 120 may terminate near the wound edge 104/114, and perforations may be introduced extending the slits 120 all the way to wound edge 104/114. The perforations will not separate during the application process and the integrity of the wound edge 104/114 will be maintained. However, in the weeks following application, due to flexion and stretching, the perforations may break at the wound edge 104/114 thereby allowing adhering subdomains of the adhesive-backed anchoring member 102/112 to operate more independently.

At the edge of the adhesive-backed anchoring member 102/112 generally opposite the wound edge 104/114, the slits 120 may be extended to the edge by perforation, as discussed above in connection with perforations approaching the wound edge 104/114. Furthermore, the integrity of the edge, generally opposite wound edge 104/114, is not as critical as the integrity of wound edge 104/114. Hence, the slits 120 may be introduced in the manufacturing process to sever the edge of the adhesive-backed anchoring member 102/112 generally opposite wound edge 104/114.

### Release Liners

The adhesive-backed surfaces of the medical device 100 may be protected (e.g., from contamination and oxidation) by the application of release liners during the manufacturing process. In some instances, multiple release liners, or release liner systems, may be used to protect a single, uninterrupted, adhesive-backed surface. A plurality of release liners may be positioned on each adhesive-backed anchoring member 102/112. In some embodiments, a first release liner, which protects the wound edge portion of an anchoring member, is removed first during the application process. In this way, the wound edge 104/114 can be adhered to the skin while leaving a protected portion of the adhesive-backed anchoring member 102/112 which can be held without the medical device 100 adhering to the fingers of the user. Once the wound edge has been applied, the second release liner can be removed to fully secure the adhesive-backed anchoring member 102/112. Illustrative release liner stock includes paper, cardboard, or polymeric sheet stock. To minimize confusion for new users of the medical device 100, the release liners may also be coded. Release liner colors or printed indicia on the release liner are examples of coding enabling a user to readily identify the order of release liner removal.

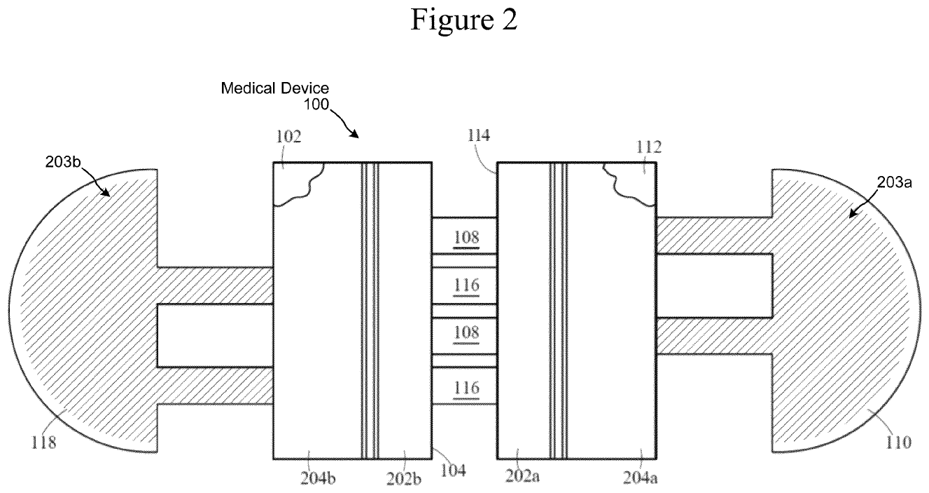

FIG. 2 is a bottom projection view of the medical device 100 showing adhesive-backed surfaces, of the adhesive-backed anchoring members 102/112, being protected by first release liners 202, second release liners 204, and third release liners 203 (shown in cross-hatching).

The medical device may include a third release liner 203a adhered to the elongated connectors 108, with the third release liner 203a having a tab portion that extends along the first pulling element 110 and which the user may pull on to remove the third release liner 203a. Likewise, a third release liner 203b may be adhered to the elongated connectors 116, with the third release liner 203b having a tab portion that extends along the second pulling element 118 and which the user may pull on to remove the third release liner 203b.

FIG. 3 is a perspective view of the medical device 100 showing the first adhesive-backed anchoring member 102 protected by the first release liner 202a and second release liner 204a. The second adhesive-backed anchoring member 112 is shown with a mirror image release liner configuration including a first release liner 202b and second release liner 204b.

Each of the release liners 202a and 204a as shown in FIG. 3 include an adhered portion (or protecting portion) in contact with and protecting an adhesive backed portion of first adhesive-backed anchoring member 102. Each of release liners 202b and 204b as shown in FIG. 3 include an adhered portion (or protecting portion) in contact with and protecting an adhesive backed portion of second adhesive-backed anchoring member 112. In FIG. 3, the adhered portion of the first release liner 202a has been assigned reference numeral 302a and the adhered portion of the second release liner 204a has been assigned reference numeral 304a. Similarly, the adhered portion of the first release liner 202b has been assigned reference numeral 302b and the adhered portion of the second release liner 204b has been assigned reference numeral 304b.

Each of the release liners shown in FIGS. 3 (202a, 204a, 202b, and 204b) also include an unadhered tab portion that extends beyond the adhesive-backed surfaces and facilitates removal of the associated release liner during application of the medical device 100. In FIG. 3, the unadhered tab portion of first release liner 202a has been assigned reference numeral 306a and the unadhered tab portion of second release liner 204a has been assigned reference numeral 308a. Similarly, the unadhered tab portion of first release liner 202b has been assigned reference numeral 306b and the unadhered tab portion of second release liner 204b has been assigned reference numeral 308b.

Tabs, or tab portions as used herein, can refer to tabs which are continuous with the associated release liner. In other words, they are produced from the same sheet stock, typically in a die cut process. Alternatively, a tab can be produced from separate stock material and attached to the release liner that it is associated with, using adhesive, for example.

As shown in FIG. 3, the first adhesive-backed anchoring member 102 has a boundary axis 310a that is parallel with the wound edge 104. Similarly, the second adhesive-backed anchoring member 112 has a boundary axis 310b that is parallel with the wound edge 114. FIG. 3 also shows that the first adhesive-backed anchoring member 102 has a boundary axis 312a that is parallel with the wound edge 104. Similarly, the second adhesive-backed anchoring member 112 has a boundary axis 312b that is parallel with the wound edge 114. Each boundary axis (310a, 310b, 312a, and 312b) defines a boundary of the adhered portion of an individual release liner. More specifically, the boundary defined by each boundary axis is an internal boundary (i.e., a boundary internal to either the first or the second adhesive-backed anchoring member). To be specific, boundary axis 310a defines an internal boundary on the first adhesive-backed anchoring member 102 that is protected by the adhered portion 302a of the first release liner 202a. Boundary axis 312a defines an internal boundary on the first adhesive-backed anchoring member 102 that is protected by adhered portion 304a of the second release liner 204a. Boundary axis 310b defines an internal boundary on the second adhesive-backed anchoring member 112 that is protected by adhered portion 302b of the first release liner 202b. Finally, boundary axis 312b defines an internal boundary on the second adhesive-backed anchoring member 112 that is protected by adhered portion 304b of the second release liner 204b.

As already mentioned, the boundary axes are parallel to a wound edge. Boundary axes 310a and 312a are parallel to the wound edge 104, and boundary axes 310b and 312b are parallel to the wound edge 114. Furthermore, because the entire adhesive-backed surface of each adhesive-backed anchoring member is completely protected by the pair of release liners, the two boundary axes associated with each adhesive-backed anchoring member must be adjacent (because the entire adhesive-backed portion of each adhesive-backed anchoring member is protected).

In use and referring to FIG. 3, the first release liners 202a and 202b may be removed and the medical device 100 subsequently applied to the skin. Once applied, the second release liners 204a and 204b may be removed so the remainder of the adhesive-backed anchoring members 102 and 112 can be adhered to the patient's skin.

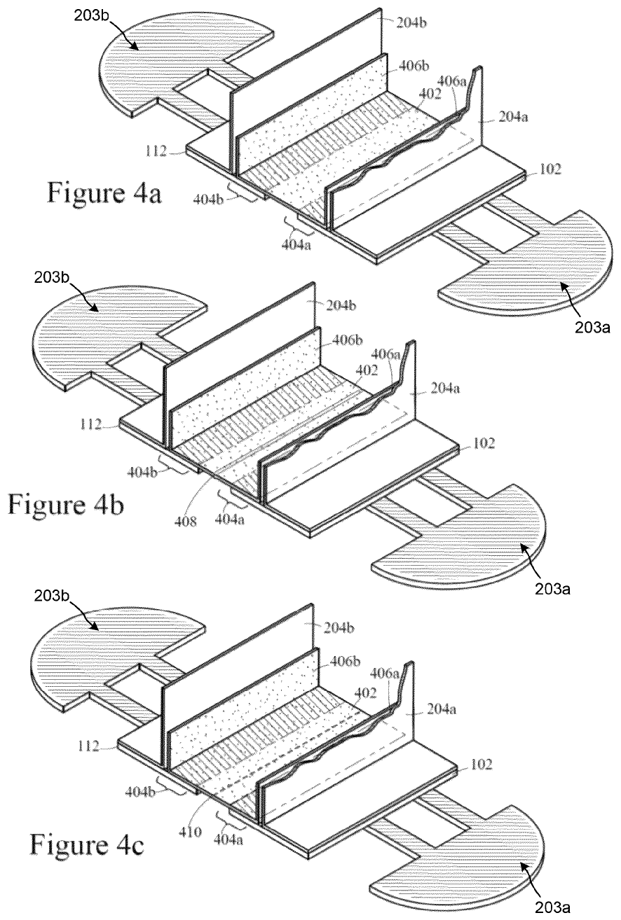

FIG. 4a shows diagrammatically an alternative instance of the present disclosure wherein a common release liner 402 (shown in stippling) is shared between the two components of the medical device 100. In particular, FIG. 4a is a diagrammatic representation showing the first adhesive-backed anchoring member 102 protected by a first adhered portion 404a (cross-hatching) of common release liner 402, and second release liner 204a. The adhesive-backed portion of second adhesive-backed anchoring member 112 is protected by a second adhered portion 404b (cross-hatching) of common release liner 402, and second release liner 204b. Common release liner 402 is provided with a first tab 406a and a second tab 406b. As depicted in the instance shown in FIG. 4a, tabs 406a and 406b are analogous to tabs 306a and 306b as discussed above in connection with FIG. 3. The transition from second adhered portions 404a and 404b of common release liner 402, to tabs 406a and 406b, respectively, are marked by a fold (not numbered).

In use and referring to FIG. 4a, the common release liner 402 may be removed and the medical device 100 subsequently applied to the skin. Once applied, the second release liners 204a and 204b may be removed so the remainder of the adhesive-backed anchoring members 102 and 112 can be adhered to the patient's skin.

Alternative instances including a common release liner 402 divided into two zones are shown diagrammatically in FIGS. 4b and 4c. The purpose of introducing two zones into the two-zone instances is to enable a user to more easily apply the medical device 100.

Referring to FIG. 4b, a crease 408 is provided in common release liner 402. Following removal of first adhered portion 404a from first adhesive-backed anchoring member 102, common release liner folds, or is folded along crease 408. Crease 408 provides a convenient feature for a user to “fold away” the loose tail of common release liner 402 while applying the adhesive portion of first adhesive-backed anchoring member 102 exposed by removal of first adhered portion 404a. Depending upon the depth of crease 408, the feature may enable a user to tear off a portion of the loose tail of common release liner along the crease, rather than merely folding it back. Crease 408 is located in a central region of common release liner 402 located between adhered portions 404a and 404b.

Referring to FIG. 4c, a frangible line 410 is provided in common release liner 402. Following removal of first adhered portion 404a from first adhesive-backed anchoring member 102, the loose tail portion is removed by fracturing common release liner 402 along frangible line 410. Frangible line 410 provides a convenient feature enabling a user to remove the loose tail of common release liner 402 while applying the adhesive portion of first adhesive-backed anchoring member 102 exposed by removal of first adhered portion 404a. Frangible line 410 is located in a central region of common release liner 402 located between adhered portions 404a and 404b.

### Release Liner Tape

The medical device 100 may include one or more instances of release liner tape removably coupled to at least one release liner and a pulling element of the medical device 100 in a manner that promotes proper removal of at least one release liner while also maintaining a relative position of the first and second components (and more particularly the first and second adhesive-backed anchoring members 102/112) of the medical device 100.

The medical device 100 may include a release liner tape 506 having an adhesive-backed surface that is removably coupled to the second release liner 204b, the third release liner 203b, and the surface of the second pulling element 118. As illustrated, the release liner tape 506 may be circular in shape and sized to not extend to any edge of the second pulling element 118.

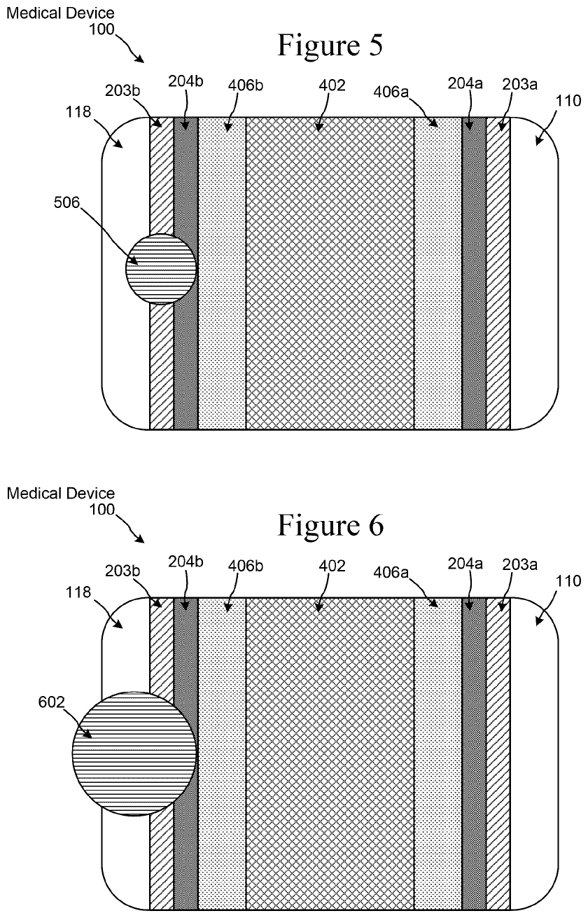

As can be seen in FIG. 5, the release liner tape 506 is not adhered to the second tab 406b of the common release liner 402. Since the release liner tape 506 is removably coupled (i.e., adhered) to portions of the first and second components of the medical device 100 (i.e., the second release liner 204b on the first adhesive-backed anchoring member 102 of the first component of the medical device 100, the third release liner 203b on the elongated connectors 116 of the second pulling element 118 of the second component of the medical device 100, and the surface of the second pulling element 118), the release liner tape 506 is configured to maintain a relative position of the first adhesive-backed anchoring member 102 with respect to the second adhesive-backed anchoring member 112 while and after the common release liner 402 is removed.

FIG. 6 illustrates another example configuration of a release liner tape 602. The release liner tape 602 has an adhesive-backed surface that is removably coupled to the second release liner 204b, the third release liner 203b, and the surface of the second pulling element 118. In contrast to the release liner tape 506 of FIG. 5, the release liner tape 602 of FIG. 6 is sized to extend beyond an edge of the second pulling element 118. The portion of the release liner tape 602 extending beyond the edge of the second pulling element 118 provides the user with an area of the release liner tape 602 that can be easily gripped and pulled on to remove the release liner tape 602 from the medical device 100.

In some embodiments, the bottom surface, of the portion of the release liner tape 602 extending beyond the edge of the second pulling element 118, may not have adhesive thereon. In other embodiments, the bottom surface, of the portion of the release liner tape 602 extending beyond the edge of the second pulling element 118, may have adhesive thereon. This may be due to case of manufacturing.

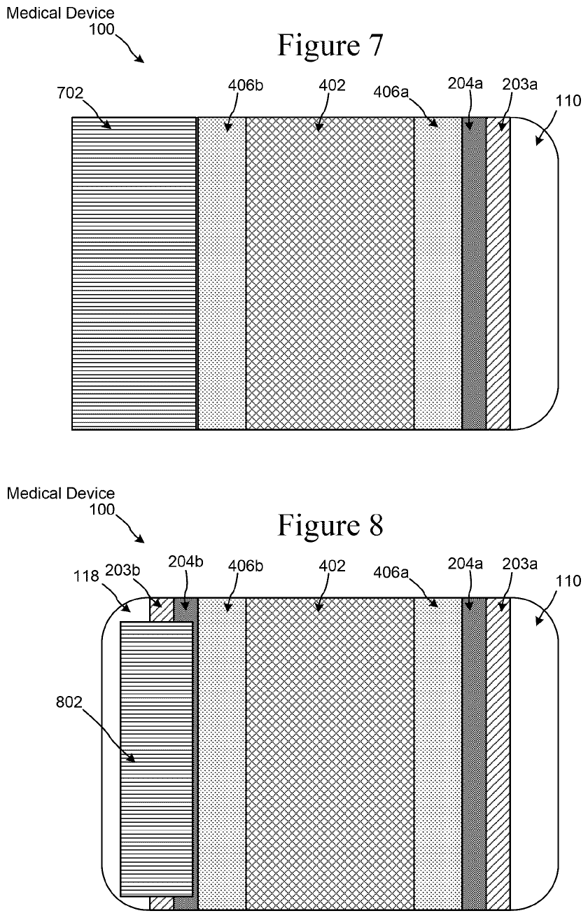

FIG. 7 illustrates another example configuration of a release liner tape 702. The release liner tape 702 has an adhesive-backed surface that is removably coupled to the second release liner 204b, the third release liner 203b, and the surface of the second pulling element 118. The release liner tape 702 is sized in one direction to be at least the length of the medical device 100 along a wound edge of the medical device 100 and is sized in an orthogonal direction to extend beyond an edge of the second pulling element 118. The portion(s) of the release liner tape 702 extending beyond the edge(s) of the second pulling element 118 provides the user with an area(s) of the release liner tape 702 that can be easily gripped and pulled on to remove the release liner tape 702 from the medical device 100.

In some embodiments, the bottom surface(s), of the portion(s) of the release liner tape 702 extending beyond the edge(s) of the second pulling element 118, may not have adhesive thereon. In other embodiments, the bottom surface(s), of the portion(s) of the release liner tape 702 extending beyond the edge(s) of the second pulling element 118, may have adhesive thereon. This may be due to case of manufacturing.

FIG. 8 illustrates another example configuration of a release liner tape 802. The release liner tape 802 has an adhesive-backed surface that is removably coupled to the second release liner 204b, the third release liner 203b, and the surface of the second pulling element 118. In contrast to the release liner tape 702 of FIG. 7, while the release liner tape 802 is elongate the release liner tape 802 is sized to not extend beyond any edge of the second pulling element 118.

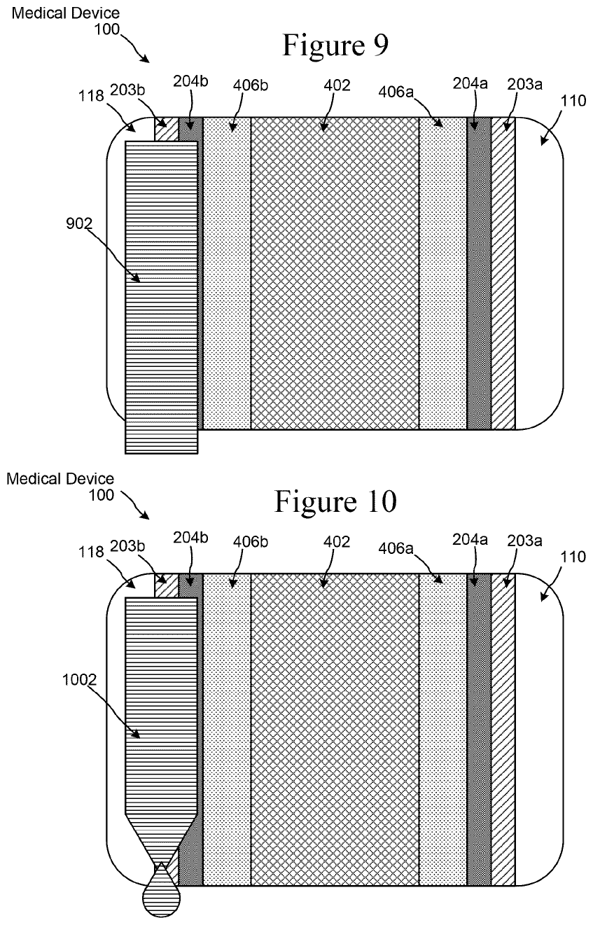

FIG. 9 illustrates another example configuration of a release liner tape 902. The release liner tape 902 has an adhesive-backed surface that is removably coupled to the second release liner 204b, the third release liner 203b, and the surface of the second pulling element 118. The release liner tape 902 is elongate and positioned to extend beyond an edge of the medical device 100 oriented orthogonal to a wound edge of the medical device 100, while not being sized to extend beyond any other edge of the medical device 100. The portion of the release liner tape 902 extending beyond the edge of the medical device 100 provides the user with an area of the release liner tape 902 that can be easily gripped and pulled on to remove the release liner tape 902 from the medical device 100.

In some embodiments, the bottom surface, of the portion of the release liner tape 902 extending beyond the edge of the medical device 100, may not have adhesive thereon or may have a dead zone (i.e., not sticky) of adhesive thereon. Various adhesive deadening techniques are known in the art/industry, any one or more of which may be used in accordance with the present disclosure. In other embodiments, the bottom surface, of the portion of the release liner tape 902 extending beyond the edge of the medical device 100, may have adhesive thereon. This may be due to case of manufacturing.

FIG. 10 illustrates another example configuration of a release liner tape 1002. The force required to de-adhere the second release liner 204b and the third release liner 203b from the medical device 100 is least at the minor edge of the second release liner 204b and the third release liner 203b. To facilitate removal of the release liner tape 1002 without such also removing the second release liner 204b and the third release liner 203b, the release liner tape 1002 may include a tapered portion that decreases in width as it approaches the edge of the medical device 100 (e.g., the minor edges of the second release liner 204b and the third release liner 203b. Such permits the release liner tape 1002 to minimally (but still beneficially) adhere to (minor edge) portions of the second release liner 204b and third release liner 203b requiring the least amount of force to remove same from the medical device 100, but also permits significant adherence of the release liner tape 1002 to (non-minor edge) portions of the second release liner 204b and third release liner 203b that require more force to remove same from the medical device 100. In some embodiments, the width of the release liner tape 1002 may taper such that a terminal point of the tapered portion extends beyond the edge of the medical device 100. As illustrated in FIG. 10, in other embodiments the width of a release liner tape 1002 may taper and then expand back out (e.g., to a circular “tear drop”) gripping portion that extends beyond the edge of the medical device 100. The gripping portion may lack adhesive thereon or may have deadened adhesive thereon. It will be appreciated that the portion of a release liner tape 1002, extending beyond the edge of the medical device 100, may be of a different size and/or shape than the remainder of the release liner tape.

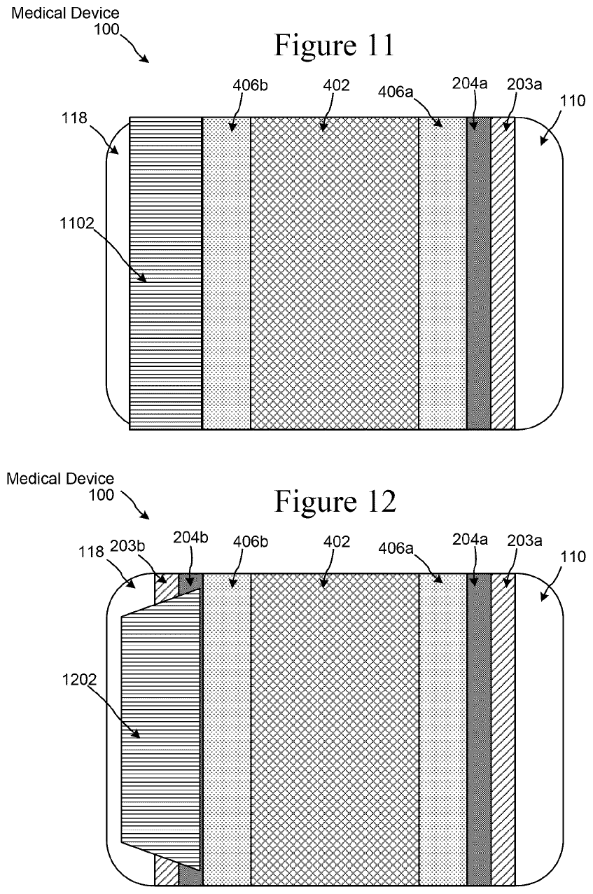

FIG. 11 illustrates another example configuration of a release liner tape 1102. The release liner tape 1102 has an adhesive-backed surface that is removably coupled to the second release liner 204b, the third release liner 203b, and the surface of the second pulling element 118. The release liner tape 1102 is sized in one direction to be (substantially) equal to the length of the medical device 100 along a wound edge of the medical device 100 and is sized in an orthogonal direction to not extend beyond an edge of the second pulling element 118.

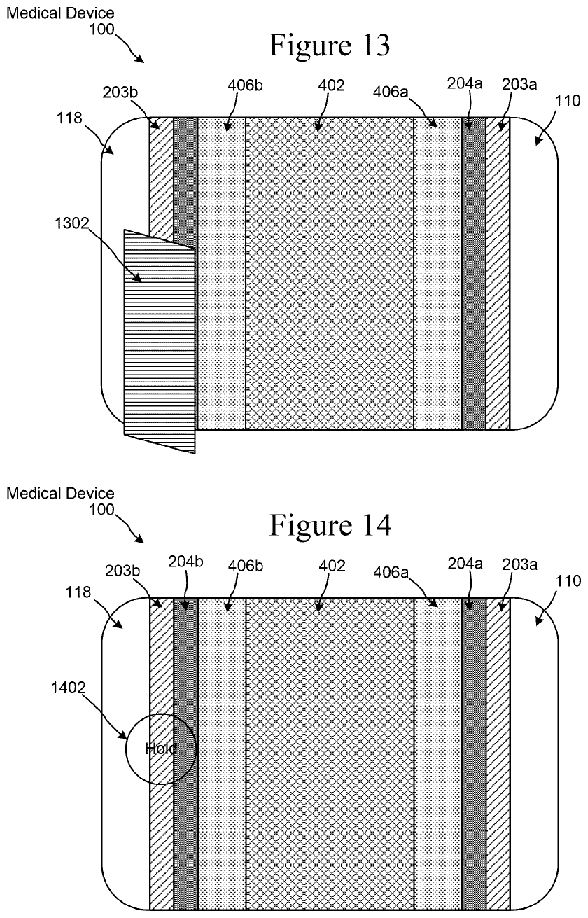

In some embodiments, the release liner tape may be configured to remove the second release liner 204b from the medical device 100 as the release liner tape is removed from the medical device 100. For example, the release liner tape may cover at least a portion of the second release liner 204b, at least a portion of the third release liner 203b, and at least a portion of the surface of the second pulling element 118, and the shape of the release liner tape and/or the areas of its bottom surface having adhesive thereon and/or the make-up of the adhesive may be configured in such a way that removal of the release liner tape from the medical device 100 simultaneously removes the second release liner 204b from the medical device 100 without also removing the third release liner 203b (which is adhered to the second pulling element 118) from the medical device 100. Using FIG. 12 as an example, a release liner tape 1202 could be trapezoidal; of its parallel sides, the longer side may be positioned on the second release liner 204b and the shorter side is positioned on the surface of the second pulling element 118 (or extends off the second pulling element 118); and only the portion of the release liner tape 1202 contacting the second release liner 204b may have adhesive (or non-deadened adhesive) thereon. Using FIG. 13 as a further example, a release liner tape 1302 may be rhomboid and positioned such that adhesive along a substantial thickness of the release liner tape 1302 adheres to a minor edge (requiring the least adhesive force to remove) of the second release liner 204b and a minor thickness (e.g., point) of the rhomboid-shaped release liner tape 1302 adheres to a portion of the third release liner 203b requiring substantial adhesive force to remove it from the medical device 500. In practice, the user would grip the portion of the release liner tape 1302 near the edge of the medical device 100 and pull the release liner tape 1302 along the major length of the release liner tape 1302, thereby simultaneously removing the second release liner 204b but not the third release liner 203b.

The adhesive backing of a release liner tape of the present disclosure may contain one or more adhesives. For example, the adhesive backing may include tackified acrylic adhesive, pressure-sensitive tackified acrylic adhesive, non-tackified acrylic adhesive, acrylate adhesive, pressure-sensitive acrylate adhesive, natural rubber adhesive, synthetic rubber adhesive, silicone adhesive (repositionable or not), heat-activated adhesive, pressure-sensitive silicone adhesive, transparent adhesive, rubber/acrylic adhesive, or a combination thereof.

A release liner tape of the present disclosure is not adhered to any portion of the common release liner 402. Since a release liner tape of the present disclosure is removably coupled (i.e., adhered) to portions of the first and second components of the medical device 100 (e.g., the second release liner 204b on the first adhesive-backed anchoring member 102 of the first component of the medical device 100, the third release liner 203b on the elongated connectors 116 of the second pulling element 118 of the second component of the medical device 100, and the surface of the second pulling element 118), the release liner tape is configured to maintain a relative position of the first adhesive-backed anchoring member 102 with respect to the second adhesive-backed anchoring member 112 while and after the common release liner 402 is removed.

In some embodiments, the third release liner 203b may not extend beyond the second release liner 204b when they are laid planar along the second pulling element 118. In such embodiments, the release liner tape 506/602/702/802/902/1002/1102/1202/1302 may removably couple (e.g., adhere) to the second pulling element 118 and the second release liner 204b, but not the third release liner 203b. Even in this configuration, the release liner tape 506/602/702/802/902/1002/11021202/1302 is able to maintain a relative position of the first adhesive-backed anchoring member 102 with respect to the second adhesive-backed anchoring member 112 while and after the common release liner 402 is removed since the second pulling element 118 and second release liner 204b are on different of the two components of the medical device 100 (i.e., the second release liner 204b is on the first adhesive-backed anchoring member 102 of the first component of the medical device 100 and the second pulling element 118 is part of the second component of the medical device 100).

In some embodiments, a portion of the bottom surface of the release liner tape 506/602/702/802/902/1002/1102/1202/1302, covering a portion of the second pulling element, may not have any adhesive thereon or may have a dead zone (i.e., not sticky) of adhesive thereon. This may enable a user to more easily remove the release liner tape 506/602/702/802/902/1002/11021202/1302 from the medical device 100.

In some embodiments, a release liner tape of the present disclosure may be positioned on the medical device 100 such that a portion of the release liner tape is covered by the second tab 406b when the second tab 406b is oriented towards and (substantially) planar with the second pulling element 118. The bottom surface, of the portion of the release liner tape covered by the second tab 406b, may not have any adhesive thereon or may have a dead zone (i.e., not sticky) of adhesive thereon. This may enable a user to more easily remove the release liner tape from the medical device 100 and/or provide the user with something to grasp on to when applying the medical device 100 to the patient's skin.

In some embodiments, a release liner tape of the present disclosure may be positioned on the medical device 100 such that a portion of the release liner tape abuts (e.g., comes up to) but is not covered by the second tab 406b when the second tab 406b is oriented towards and (substantially) planar with the second pulling element 118.

Various adhesive deadening techniques are known in the art/industry, any one or more of which may be used in accordance with the present disclosure.

While FIGS. 5-13 illustrate the medical device 100 having release liner tape removably coupled (e.g., adhered) to the second release liner 204b, the third release liner 203b, and the surface of the second pulling element 118, it will be appreciated that a similarly configured release liner tape may additionally or alternatively be removably coupled to the second release liner 204a, the third release liner 203a, and the surface of the first pulling element 110.

While FIGS. 5-13 illustrate release liner tapes of specific sizes and shapes, it will be appreciated that the size and shape of a release liner tape may differ from that illustrated provided the release liner tape is configured to removably couple and function as described herein.

### Release Liner Removal Holding Indicia

The medical device 100 may include one or more indicia for holding a release liner(s) while removing another release liner of the medical device 100. The indicia may be located on at least two release liners of the medical device 100 in a manner that promotes proper removal of at least one release liner while also maintaining a relative position of the first and second components (and more particularly the first and second adhesive-backed anchoring members 102/112) of the medical device 100.

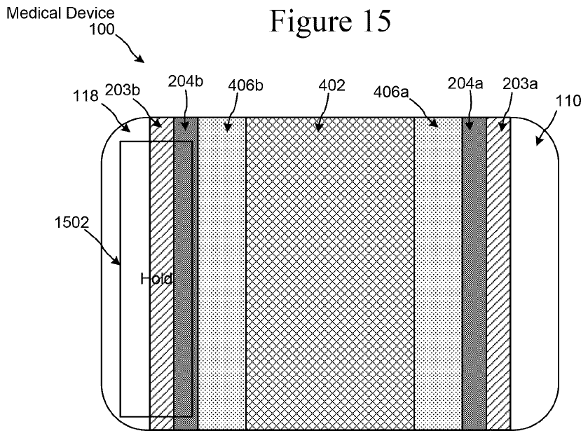

Referring to FIGS. 14 and 15, the medical device 100 may include indicia 1402/1502 located (e.g., printed) on the second release liner 204b, the third release liner 203b, and the surface of the second pulling element 118. Although FIG. 14 shows the indicia 1402 as being circular and FIG. 15 shows the indicia 1502 as being rectangular, the indicia 1402/1502 need not necessarily be of any particular shape.

The indicia 1402/1502 may be printed on the surfaces of the second release liner 204b, the third release liner 203b, and the surface of the second pulling element 118.

Since the indicia 1402/1502 is located on portions of the first and second components of the medical device 100 (i.e., the second release liner 204b on the first adhesive-backed anchoring member 102 of the first component of the medical device 100, the third release liner 203b on the elongated connectors 116 of the second pulling element 118 of the second component of the medical device 100, and the surface of the second pulling element 118), the indicia 1402/1502 is designed to facilitate a user to simultaneously grasp aspects of the first and second components of the medical device 100 and thereby maintain a relative position of the first adhesive-backed anchoring member 102 with respect to the second adhesive-backed anchoring member 112 while and after the common release liner 402 is removed.

It is noted that the indicia 1402/1502 is not located on any portion of the common release liner 402. Since the indicia 1402/1502 is located on portions of the first and second components of the medical device 100 (e.g., the second release liner 204b on the first adhesive-backed anchoring member 102 of the first component of the medical device 100, the third release liner 203b on the elongated connectors 116 of the second pulling element 118 of the second component of the medical device 100, and the surface of the second pulling element 118) without being located on the common release liner 402, the indicia 1402/1502 facilitates a user to simultaneously grasp aspects of the first and second components of the medical device 100 to thereby maintain a relative position of the first adhesive-backed anchoring member 102 with respect to the second adhesive-backed anchoring member 112 while and after the common release liner 402 is removed.

In some embodiments, the third release liner 203b may not extend beyond the second release liner 204b when they are laid planar along the second pulling element 118. In such embodiments, the indicia 1402/1502 may be located on the second pulling element 118 and the second release liner 204b, but not the third release liner 203b. Even in this configuration, the indicia 1402/1502 is able to facilitate the simultaneous grasping of the first and second components of the medical device 100 by the user such that a relative position of the first adhesive-backed anchoring member 102 with respect to the second adhesive-backed anchoring member 112 is maintained while and after the common release liner 402 is removed since the second pulling element 118 and second release liner 204b are on different of the two components of the medical device 100 (i.e., the second release liner 204b is on the first adhesive-backed anchoring member 102 of the first component of the medical device 100 and the second pulling element 118 is part of the second component of the medical device 100).

While FIGS. 14-15 illustrate the medical device 100 having the indicia 1402/1502 located on the second release liner 204b, the third release liner 203b, and the surface of the second pulling element 118, it will be appreciated that similar indicia may additionally or alternatively be located on the second release liner 204a, the third release liner 203a, and the surface of the first pulling element 110.

### Methods of Closing a Wound or Incision

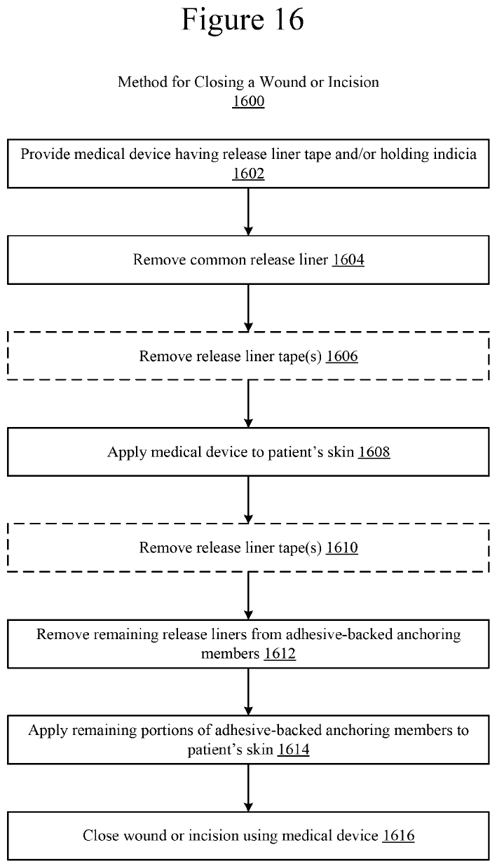

The present disclosure also provides methods for closing wounds and incisions. An example method 1600 is illustrated in FIG. 16.

The method 1600 includes providing (step 1602) a medical device 100 having release liner tape 506/602/702/802/902/1002/11021202/1302 and/or holding indicia 1402/1502.

After providing the medical device 100, the method 1600 includes removing (step 1604) the common release liner 402.

Optionally, after removing the common release liner 402 the method 1600 may include removing (step 1606) one or more release liner tapes from the medical device 100.

After removing the common release liner 402 [and optionally after removing the release liner tape(s)], the method 1600 includes applying (step 1608) the medical device 100 to the patient's skin. Removing the common release liner 402 results in portions of the adhesives of the adhesive-backed anchoring members 102 and 112 becoming exposed. These portions of the adhesives may be applied to the patient's skin proximate to opposing edges of the wound or incision. In embodiments where the medical device 100 includes a release liner tape positioned on the medical device 100 such that a portion thereof is covered by the second tab 406b when the second tab 406b is oriented towards and (substantially) planar with the second pulling element 118, and the bottom surface of the portion of the release liner tape covered by the second tab 406b does not have any adhesive thereon or has a dead zone of adhesive, applying the medical device 100 to the patient's skin may be done while grasping the first pulling element 110 in one hand and in the other hand grasping the portion of the release liner tape not having any adhesive thereon or having a dead zone of adhesive.

Optionally, after applying the medical device 100 to the patient's skin, the method 1600 may include removing (step 1610) one or more release liner tapes from the medical device 100 (e.g., when step 1606 is not performed).

After the medical device 100 is applied to the patient's skin at step 1608, the method 1600 may include removing (step 1612) the remaining release liners from the adhesive-backed anchoring members and applying (step 1614) the remaining portions of the adhesive-backed anchoring members to the patient's skin. In some embodiments where steps 1606 and 1610 are not performed, step 1612 of the method 1600 may include removing the release liner tape(s) removed in conjunction with removing one or more of the remaining release liners.

Thereafter, the method 1600 may proceed with closing (step 1616) the wound or incision using the medical device 100 as discussed elsewhere herein.

### Further Definitions and Construction of Terms

The titles, headings, and subheadings provided herein should not be interpreted as limiting the various aspects of the disclosure. Accordingly, the terms defined herein are more fully defined by reference to the specification in its entirety. All references cited herein are incorporated by reference in their entirety.

Unless otherwise defined, scientific and technical terms used herein shall have the meanings that are commonly understood by those of ordinary skill in the art. Further, unless otherwise required by context, singular terms shall include pluralities, and plural terms shall include the singular.

In this application, the use of “or” means “and/or” unless stated otherwise. In the context of a multiple dependent claim, the use of “or” refers back to more than one preceding independent or dependent claim in the alternative only.

It is further noted that, as used in this specification and the appended claims, the singular forms “a,” “an,” and “the,” and any singular use of any word, include plural referents unless expressly and unequivocally limited to one referent.

As used herein, the term “about,” means approximately. When the term “about” is used in conjunction with a numerical range, it modifies that range by extending the boundaries above and below the numerical values set forth. Illustratively, the use of the term “about” indicates that values slightly outside the cited values (i.e., plus or minus 0.1% to 10%), which are also effective and safe are included in the value. Numerical ranges recited herein by endpoints include all numbers and fractions subsumed within that range (e.g. 1 to 5 includes 1, 1.5, 2, 2.75, 3, 3.90, 4, and 5).

As used herein, the terms “comprising” (and any form of comprising, such as “comprise,” “comprises,” and “comprised”), “having” (and any form of having, such as “have” and “has”), “including” (and any form of including, such as “includes” and “include”), and “containing” (and any form of containing, such as “contains” and “contain”) are inclusive or open-ended and do not exclude additional, un-recited elements or method steps. Additionally, a term that is used in conjunction with the term “comprising” is also understood to be able to be used in conjunction with the term “consisting of” or “consisting essentially of.”

Method steps described in this disclosure can be performed in any order unless otherwise indicated or otherwise clearly contradicted by context.

For the avoidance of doubt, insofar as is practicable any embodiment of a given aspect of the present disclosure may occur in combination with any other embodiment of the same aspect of the present disclosure. In addition, insofar as is practicable it is to be understood that any preferred or optional embodiment of any aspect of the present disclosure should also be considered as a preferred or optional embodiment of any other aspect of the present disclosure.

What is claimed is:

1. A medical device for closing a wound or incision, comprising:
first and second components that are movable with respect to each other for closing a wound or incision;
a first release liner removably adhered to a first adhesive-backed surface of the first component;
a second release liner removably adhered to a second adhesive-backed surface of the first component and/or a first adhesive-backed surface of the second component; and
means located on the first release liner and the second component for maintaining a relative position of the first and second components while and after the second release liner is removed from the medical device.

2. The medical device of claim 1, wherein the means does not extend to any edge of the second component.

3. The medical device of claim 1, wherein the means is not located on the second release liner.

4. The medical device of claim 1, wherein the means is a release liner tape.

5. The medical device of claim 4, wherein the release liner tape extends beyond at least one edge of the second component.

6. The medical device of claim 5, wherein a portion of the release liner tape, extending beyond the at least one edge of the second component, does not have any adhesive thereon.

7. The medical device of claim 5, wherein a portion of the release liner tape, extending beyond the at least one edge of the second component, has a deadened adhesive thereon.

8. The medical device of claim 5, wherein the release liner tape extends beyond an edge of the second component oriented orthogonal to a wound edge of the second component.

9. The medical device of claim 5, wherein the release liner tape extends beyond an edge of the second component oriented parallel to a wound edge of the second component.

10. The medical device of claim 4, wherein the release liner tape decrease in width as it moves toward an edge of the medical device.

11. The medical device of claim 4, wherein the release liner tape is configured to remove the first release liner from the medical device when the release liner tape is removed from the medical device.

12. The medical device of claim 4, wherein a bottom surface of portion of the release liner tape, covering a portion of a pulling element of the first component, either lacks adhesive thereon or has a dead zone of adhesive thereon.

13. The medical device of claim 4, wherein:
a portion of the release liner tape is covered by a tab of the second release liner when the tab is oriented towards and planar with a pulling element of the first component; and
a bottom surface, of the portion of the release liner tape covered by the tab, either lacks adhesive thereon or has a dead zone of adhesive thereon.

14. The medical device of claim 1, wherein the means is located on the first release liner and a pulling element of the second component, the pulling element being configured to receive a pulling force to close the wound or incision.

15. The medical device of claim 1, wherein the means is indicia printed on the first release liner and the second component.

16. The medical device of claim 1, wherein the first adhesive-backed surface of the first component is on an adhesive-backed anchoring member of the first component, and the adhesive-backed anchoring member is configured to be adhered to skin proximate the wound or incision.

17. The medical device of claim 1, further comprising a third release liner removably adhered to a second adhesive-backed surface of the second component, wherein the means is located on the first release liner, the second component, and the third release liner.

18. The medical device of claim 17, wherein the second adhesive-backed surface of the second component is on an elongated connector for adhering the first component to the second component and maintaining the wound or incision in a closed state.

19. The medical device of claim 1, wherein the second release liner is removably adhered to the second adhesive-backed surface of the first component and the first adhesive-backed surface of the second component.

20. The medical device of claim 19, wherein:
the second adhesive-backed surface of the first component is on an adhesive-backed anchoring member of the first component;
the first adhesive-backed surface of the second component is on an adhesive-backed anchoring member of the second component; and
the adhesive-backed anchoring members are configured to be adhered to skin proximate the wound or incision.

21. A method for closing a wound or incision, comprising:
providing the medical device of claim 1;
applying the medical device to skin of a patient proximate the wound or incision; and
after applying the medical device, closing the wound or incision using the medical device.
